(19) United States
(12) Patent Application Publication
(10) Pub. No.: US 2006/0177406 A1
(43) Pub. Date: Aug. 10, 2006

(54) Formula, a system and a method for treating urushiol induced contact dermatitis

(76) Inventors: Sarfaraz Khan Niazi, Deerfield, IL (US); Nestor Sebastian Tomasi, Joliet, IL (US)

Correspondence Address: PATENTS+TMS, P.C., 2849 W. ARMITAGE AVE., CHICAGO, IL, 60647, US

(21) Appl. No.: 11/053,400

(22) Filed: Feb. 8, 2005

(51) Int. Cl. A61K 8/42; A61K 8/27

(52) U.S. Cl. 424/70.22; 424/401

(57) ABSTRACT

A formula, a system and a method is provided for treating urushiol induced contact dermatitis, chemical irritants and/or microbiological irritants on an affected area of skin of a user. The formula and/or the system is applied to an area of the skin of the user affected with urushiol, the chemical irritants and/or the microbiological irritants. The formula and/or the system have a cleansing agent, one or more surfactants and/or one or more abrasive agents for treating the urushiol induced contact dermatitis, the chemical irritants and/or the microbiological irritants. One or more abrasive agents separates the urushiol, the chemical irritants and/or the microbiological irritants from the skin of the user at the affected area. The cleansing agent bonds to the urushiol, the chemical irritants and/or the microbiological irritants at the affected area and withdraws the urushiol, the chemical irritants and/or the microbiological irritants from the tissue under the affected area of the skin of the user. The formula, the system, the urushiol, the chemical irritants and/or the microbiological irritants may be rinsed away from and/or may be removed from the affected area of the skin of the user. As a result, the urushiol induced contact dermatitis, the chemical irritants and/or the microbiological irritants is treated and/or is terminated by the formula, the system, the cleansing agent and/or one or more surfactants.

### BACKGROUND OF THE INVENTION

The present invention generally relates to a formula, a system and a method for treating chemical irritants and/or microbiological irritants on skin of a user. More specifically, the present invention relates to a formula, a system and a method for treating the chemical irritants and/or the microbiological irritants which be applied to an affected area of the skin of the user. The affected area of the skin of the user may be affected with the, for example, urushiol induced contact dermatitis. The formula and/or the system may have one or more surfactants and/or a cleansing agent which may treat the urushiol induced contact dermatitis and/or may remove the chemical irritants and/or the microbiological irritants from the skin of the user. The formula and/or the system may be applied to the affected area of the skin of the user. One or more surfactants may bond to the urushiol, the chemical irritants and/or the microbiological irritants which may be located on the affected area of the skin of the user. The formula, the system and/or the urushiol, the chemical irritants and/or the microbiological irritants may be rinsed away from and/or may be removed from the affected area of the skin of the user with water, a solvent and/or the like. The affected area of the skin of the user, the urushiol induced contact dermatitis, the chemical irritants and/or the microbiological irritants may be treated by the formula, the system and/or one or more surfactants of the formula and/or the system.

It is generally known that skin of a user may be affected by dermatitis which may be transferred from a plant to the skin of the user. The dermatitis may be caused from, for example, poison ivy, poison oak, poison sumac and/or the like. The plant may have an active toxin therein which may cause the dermatitis to develop on the skin of the user. The active toxin of the plant may be transferred to an area of the skin of the user during contact of the area of the skin with the plant. The active toxin of the plant may be, for example, urushiol which may be an organic molecule. The active toxin, the organic molecule and/or the urushiol may bond to the area of the skin and/or may permeate tissue under the area of the skin of the user. As a result, the area of the skin and/or the tissue under the area of the skin may be an affected area of the skin of the user. Moreover, the active toxin on the area of the skin, the organic molecule and/or the urushiol may not be soluble and/or may not be removed with a solvent, such as, for example, water.

Traditionally, an organic solvent, such as, for example, a laundry soap may be applied to the area of the skin of the user to treat the dermatitis. The organic solvent may not reduce irritation of the affected area of skin caused by the active toxin, the organic molecule and/or the urushiol. Further, the organic solvent may spread the active toxin, the organic molecule and/or the urushiol to other areas of the skin of the user. The organic solvent may not bond to the urushiol in the affected area of the skin of the user. Further, the organic solvent may not permeate the skin of the affected area and/or the tissue under the affected area of the user as easily as the urushiol may permeate the skin and/or the tissue of the user. Still further, the organic solvent may not remove the urushiol from the affected area of the skin of the user. Moreover, the organic solvent may not withdraw the urushiol from the tissue under the affected area of the skin of the user. As a result, the organic solvent may not treat the urushiol induced contact dermatitis on the skin of the user. Furthermore, the organic solvent may not terminate and/or may not prevent the irritation at the affected area of the skin of the user which may be caused by the urushiol.

A need, therefore, exists for a formula, a system and a method for treating urushiol induced contact dermatitis, the chemical irritants and/or the microbiological irritants. Additionally, a need exists for a formula, a system and a method for treating urushiol induced contact dermatitis, the chemical irritants and/or the microbiological irritants which may be applyied to the affected area of the skin of the user. Further, a need exists for a formula, a system and a method for treating urushiol induced contact dermatitis, the chemical irritants and/or the microbiological irritants which may have one or more surfactants therein for applying to the affected area of the skin of the user. Still further, a need exists for a formula, a system and a method for treating urushiol induced contact dermatitis which provides one or more surfactants for bonding to the urushiol, the chemical irritants and/or the microbiological irritants at the affected area of the skin of the user. Moreover, a need exists for a formula, a system and a method for treating urushiol induced contact dermatitis which provides one or more surfactants for removing urushiol, the chemical irritants and/or the microbiological irritants from the affected area of the skin of the user and/or for withdrawing the urushiol from the tissue under the affected area of the skin of the user.

### SUMMARY OF THE INVENTION

The present invention generally relates to a formula, a system and a method for treating chemical irritants and/or microbiological irritants at an affected area of skin of a user. More specifically, the present invention relates to a formula, a system and a method for treating urushiol induced contact dermatitis, the chemical irritants and/or the microbiological irritants which may be applied to the area of skin affected with urushiol. The formula and/or the system may have one or more surfactants and/or cleansing agents, such as, for example, sodium cocoyl sarcosinate for treating urushiol induced contact dermatitis, the chemical irritants and/or the microbiological irritants at an area of skin of the user. Further, the formula and/or the system may have one or more abrasive agents, such as, for example, polyethylene granules and/or silica for separating and/or for dislodging the urushiol, the chemical irritants and/or the microbiological irritants from the affected area of the skin of the user. One or more surfactants in the formula and/or the system may bond to the urushiol at the affected area of the skin of the user and/or in the tissue under the affected area of the skin of the user. The formula, the system and/or the urushiol, the chemical irritants and/or the microbiological irritants may be rinsed away from, may be removed from and/or may be withdrawn from the affected area of the skin of the user and/or the tissue under the affected area of the skin of the user. Moreover, the urushiol, the chemical irritants and/or the microbiological irritants may be removed from the affected area and/or may be withdrawn from the tissue under the affected area by one or more abrasive agents and/or one or more surfactants in the formula and/or the system. As a result, the urushiol induced contact dermatitis, the chemical irritants and/or the microbiological irritants at the affected area of the skin of the user may be treated by the formula and/or the system.

To this end, in an embodiment of the present invention, a formula for treating dermatitis on an area of skin of a user wherein the area of the skin is affected with urushiol wherein the formula is applied to the area of the skin of the user is provided. The formula has a cleansing agent to bind to the urushiol at the area of the skin of the user. Further, the formula has a first surfactant to remove the urushiol from the area of the skin of the user. Moreover, the formula has a first abrasive agent to separate the urushiol from the skin of the user wherein the formula and the urushiol are rinsed from the area of the skin of the user.

In an embodiment, the first surfactant is nonoxynol-9 or C12-15 pareth-9.

In an embodiment, the cleansing agent is sodium cocoyl sarcosinate.

In an embodiment, the formula has a second surfactant.

In an embodiment, the formula has a photo-stabilizer.

In an embodiment, the first abrasive agent is silica or polyethylene granules.

In an embodiment, the formula has a second abrasive agent.

In an embodiment, the formula has a zinc oxide.

In another embodiment, a system for treating an area of skin of a user wherein the area of the skin is affected with urushiol and further wherein the formula is applied to the area of the skin of the user to remove the urushiol therefrom is provided. The system has a plurality of surfactants to bond to the urushiol wherein one of the plurality of surfactants is a cleansing agent. Further, the system has an abrasive agent to separate the urushiol from the skin of the user at the area affected with the urushiol. Moreover, the system has a photo-stabilizer to protect one of the plurality of surfactants from photo-degradation wherein the formula and the urushiol are washed from the area of the skin of the user.

In an embodiment, one of the plurality of surfactants is nonoxynol-9 or C12-15 pareth-9.

In an embodiment, the cleansing agent is sodium cocoyl sarcosinate.

In an embodiment, the abrasive agent is silica or polyethylene granules.

In an embodiment, the photo-stabilizer is a zinc oxide.

In another embodiment, a method for treating dermatitis on an area of skin of a user wherein the area of the skin of the user is affected with urushiol is provided. The method has the step of providing a formula to remove the urushiol from the area of skin of the user wherein the formula has a first surfactant, a first abrasive agent and a cleansing agent therein. Further, the method has the steps of applying a first treatment of the formula to the area of the skin of the user and allowing the cleansing agent to bond to the urushiol on the area of the skin of the user. Moreover, the method has the step of removing the formula and the urushiol from the area of the skin of the user.

In an embodiment, the method has the step of adding a photo-stabilizer to the formula.

In an embodiment, the method has the step of adding a second surfactant or a second abrasive agent to the formula.

In an embodiment, the method has the step of applying a second treatment of the formula to the area of the skin of the user.

In an embodiment, the method has the step of separating the urushiol from the area of the skin of the user with the abrasive agent.

In an embodiment, the method has the step of protecting the first surfactant from photo-degradation.

In an embodiment, the cleansing agent is sodium cocoyl sarcosinate.

It is, therefore, an advantage of the present invention to provide a formula, a system and a method for treating urushiol induced contact dermatitis.

Another advantage of the present invention is to provide a formula, a system and a method for treating urushiol induced contact dermatitis which may be applied to an area affected by urushiol.

And, another advantage of the present invention is to provide a formula, a system and a method for treating urushiol induced contact dermatitis which may have one or more surfactants for applying to an affected area of skin of a user.

Yet another advantage of the present invention is to provide a formula, a system and a method for treating urushiol induced contact dermatitis which may have one or more surfactants for removing urushiol from an affected area of skin of a user.

A further advantage of the present invention is to provide a formula, a system and a method for treating urushiol induced contact dermatitis which may have one or more surfactants for bonding to urushiol at an affected area of skin of a user and/or in tissue under the affected area of skin of the user.

Moreover, an advantage of the present invention is to provide a formula, a system and a method for treating urushiol induced contact dermatitis which may have sodium cocoyl sarcosinate for removing urushiol from the area of skin of a user and/or for withdrawing the urushiol from the tissue under the affected area of skin of the user.

And, another advantage of the present invention is to provide a formula, a system and a method for treating urushiol induced contact dermatitis which may have one or more abrasive agents for removing urushiol from an affected area of skin of a user.

Yet another advantage of the present invention is to provide a formula, a system and a method for treating urushiol induced contact dermatitis which may have one or more abrasive agents for separating urushiol from skin at an affected area of skin of a user.

Another advantage of the present invention is to provide a formula, a system and a method for treating urushiol induced contact dermatitis which may have polyethylene granules and/or silica for dislodging urushiol from skin at an affected area of skin of a user.

A further advantage of the present invention is to provide a formula, a system and a method for treating urushiol induced contact dermatitis which may have nonoxynol-9, C12-C15 Pareth-9 and/or sodium cocoyl sarcosinate for treating the urushiol induced contact dermatitis at an affected area of skin of a user and/or in tissue under the affected area of skin of the user.

Moreover, an advantage of the present invention is to provide a formula, a system and a method for treating urushiol induced contact dermatitis which may be applied to an affected area of skin of a user and may rinse away the urushiol from the affected area of the skin of the user.

And, another advantage of the present invention is to provide a formula, a system and a method for treating urushiol induced contact dermatitis which may have a bulking agent and/or a photo-stabilizer for protecting skin of the user from ultraviolet radiation.

Yet another advantage of the present invention is to provide a formula, a system and a method for treating urushiol induced contact dermatitis which may have a zinc oxide for protecting skin of the user from ultraviolet radiation.

Another advantage of the present invention is to provide a formula, a system and a method for treating urushiol induced contact dermatitis which may have nonoxynol-9, C12-C15 Pareth-9 and sodium cocoyl sarcosinate for deep cleaning an affected area of skin of a user.

A further advantage of the present invention is to provide a formula, a system and a method for treating urushiol induced contact dermatitis which may have a surfactant and zinc to remove the urushiol from an affected area of skin of a user and/or to protect the skin from absorption of the urushiol.

Additional features and advantages of the present invention are described in, and will be apparent from, the detailed description of the presently preferred embodiments and from the drawings.

### DETAILED DESCRIPTION OF THE PRESENTLY PREFERRED EMBODIMENTS

The present invention generally relates to a formula, a system and a method for treating chemical irritants and/or microbiological irritants on skin of a user. More specifically, the present invention relates to a formula, a system and a method for treating the chemical irritants and/or the microbiological irritants which ay be applied to an affected area of skin on the user. The affected area of the skin of the user may be affected with the chemical irritants and/or the microbiological irritants. The chemical irritants may be derived from an environment or from contact with, for example, plants, animals, cosmetics, clothes and/or the like. Further, the chemical irritants may be derived from an acute use, a chronic use or an exposure basis. The chemical irritants may cause a dermatitis, such as, for example, urushiol induced contact dermatitis. The dermatitis may be caused from a plant of the ginkgoaceae family or the anacardiaceae family, such as, for example, poison ivy, poison oak, poison sumac and/or the like.

The plant may have an active toxin thereon which may cause the dermatitis to develop on the skin of the user. The active toxin of the plant may be transferred to an area of the skin of the user during contact between the area of the skin with the plant. The active toxin of the plant may be, for example, urushiol which may be an organic molecule. The organic molecule may be, for example, a catechol, a di-phenol and/or the like which may be insoluble in water. Further, the organic molecule may be 1,2-dihydroxy benzene with a 15-carbon atom side-chain in the 3 position. The 15-carbon atom side-chain may have one or more degrees of unsaturation. The active toxin and/or the urushiol may bond to the area of the skin and/or may permeate tissue under the area of the skin of the user. As a result, the area of the skin and/or the tissue under the area of the skin may be an affected area of the skin of the user.

The formula and the system may be, for example, a phenol derivative which may have one or more alkyl side chains. The formula and/or the system may be structurally similar to the organic molecule and/or the urushiol. One or more alkyl side chains may be added to a phenol group by a hydrophilicity reaction with ethylene oxide. The formula and/or the system may be, for example, an alkyl aryl polyglycol ether. The formula and/or the system may be, for example, a cream, an aqueous gel, a spray and/or the like. The formula and/or the system may have one or more surfactants and/or a cleansing agent for treating the urushiol induced contact dermatitis. The cleansing agent may bond to the urushiol, the chemical irritants and/or the microbiological irritants located on the affected area of the skin of the user.

The formula and/or the system may be in an essentially moisture-free state. As a result, the formula and/or the system may dissolve the urushiol, the chemical irritants and/or the microbiological irritants at the affected area of the skin of the user. The formula and/or the system may dissolve the urushiol, the chemical irritants and/or the microbiological irritants based on a structural similarity of the alkyl aryl polyglycol ether and the urushiol, the chemical irritants and/or the microbiological irritants. The structural similarity of the alkyl aryl polyglycol ether and the urushiol, the chemical irritants and/or the microbiological irritants may enhance mutual co-solvency of the formula and/or the system and the urushiol, the chemical irritants and/or the microbiological irritants. The formula and/or the system may clean the urushiol, the chemical irritants and/or the microbiological irritants from the affected area of the skin of the user. The formula, the system and/or the urushiol, the chemical irritants and/or the microbiological irritants may be rinsed away from and/or may be removed from the affected area of the skin of the user with a solvent, such as, for example, water and/or the like. As a result, the affected area of the skin of the user and/or the urushiol induced contact dermatitis, the chemical irritants and/or the microbiological irritants may be treated by the formula, the system and/or the cleaning agent of the formula and/or the system.

The formula and/or the system may have one or more surfactants therein. One or more surfactants may be a non-aqueous surfactant. Further, one or more surfactants may be an emulsifying agent and/or a cleansing agent. The emulsifying agent may be, for example, nonoxynol-9, C12-15 pareth-9 and/or the like. A weight percentage range of 2% to 3% of a total composition of the formula and/or the system may be the emulsifying agent. The present invention should not be deemed as limited to the embodiments of a specific emulsifying agent in the formula and/or the system.

The cleansing agent may be, for example, sodium cocoyl sarcosinate and/or the like. The sodium cocoyl sarcosinate may be a derivative of coconut oil and an amino acid, such as, for example, sarcosine. The sodium cocoyl sarcosinate may be applied to the skin of the user without causing irritation and/or inflammation of the skin of the user. The sodium cocoyl sarcosinate may be mixed with an oil, such as, the urushiol, the chemical irritants and/or the microbiological irritants, to create a lather. A weight percentage range of 4% to 6% of the total composition of the formula and/or the system may be the cleansing agent. Alternatively, the cleansing agent may be, for example, sucrose cocoate, lauryl polyglucose, sodium cocoyl glutamate, sodium isostearoyl lactylate, cocamidopropyl hydroxysultaine, disodium cocoamphodiacetate, sodium lauryl sulfoacetate and/or the like.

The formula and/or the system may have one or more abrasive agents therein. One or more abrasive agents may be an absorbent agent and/or a binder agent. The absorbent agent may be, for example, silica, silica gel and/or the like. A weight percentage range of 0.05% to 0.15% of the total composition of the formula and/or the system may be the absorbent agent. The binder agent may be, for example, polyethylene granules and/or the like. The polyethylene granules may be micro polyethylene beads. A weight percentage range of 15% to 25% of the total composition of the formula and/or the system may be the binder agent. The formula and/or the system may include a combination of both the absorbent agent and the binder agent. The one or more abrasive agents may ash and/or may remove the urushiol, the chemical irritants and/or he microbiological irritants from the affected area of the skin of the patient. Further, one or more abrasive agents may bind to the urushiol, the chemical irritants and/or the microbiological irritants at the affected area of the skin of the patient. The resent invention should not be deemed as limited to the embodiments of a specific absorbent agent and/or a specific binder agent in the formula and/or the system.

The formula and/or the system may have a chelating agent therein. The chelating agent may be, for example, di-sodium ethylenediamine tetra-acetic acid and/or the like. A weight percentage range of 0.03% to 0.06% of the total composition of the formula and/or the system may be the chelating agent. The present invention should not be deemed as limited to the embodiments of a specific chelating agent in the formula and/or the system.

The formula and/or the system may have an emulsion stabilizer therein. The emulsion stabilizer may be a viscosity agent, such as, for example, a carbomer and/or the like. A weight percentage range of 1.6% to 2% of the total composition of the formula and/or the system may be the emulsion stabilizer. The present invention should not be deemed as limited to the embodiments of a specific emulsion stabilizer in the formula and/or the system.

The formula and/or the system may have a bulking agent therein. The bulking agent may be, for example, a photo-stabilizer. The bulking agent and/or the photo-stabilizer may be, for example, avobenzone, octocrilene, octyl p-methoxycinnamate, a zinc oxide and/or the like. The photo-stabilizer may protect one or more surfactants, such as, the nonoxynol-9 from photo-degradation which may produce carcinogens in the formula and/or the system. A weight percentage range of 0.12% to 0.2% of the total composition of the formula and/or the system may be the bulking agent. Further, the photo-stabilizer may protect the skin of the user from ultraviolet radiation, such as, for example, UVA radiation and/or UVB radiation. The UVA radiation and/or the UVB radiation may activate the 15-carbon side-chain of the urushiol. As a result, the UVA radiation and/or the UVB radiation may increase the irritation caused by the urushiol at the affected area of the skin of the user. The photo-stabilizer of the formula and/or the system may prevent the UVA radiation and/or the UVB radiation from activating the 15-carbon side-chain of the urushiol, the chemical irritants and/or the microbiological irritants. As a result, the photo-stabilizer of the formula and/or the system may prevent irritation caused by the urushiol at the affected area of the skin of the user. The present invention should not be deemed as limited to the embodiments of a specific bulking agent and/or a specific photo-stabilizer in the formula and/or the system.

The formula and/or the system may have a preservative therein. The preservative may be, for example, propylene glycol, diazolidinyl urea, methylparaben, propylparaben and/or the like. A weight percentage range of 0.9% to 1.1% of the total composition of the formula and/or the system may be the preservative. The propylene glycol may enhance the co-solvency of the formula and/or the system. The formula and/or the system may have a pondus hydrogenii (hereinafter “pH”) adjuster therein. The pH adjuster may be, for example, triethanolamine and/or the like. A weight percentage range of 1.5% to 2.2 of the total composition of the formula and/or the system may be the pH adjuster. The present invention should not be deemed as limited to the embodiments of a specific preservative and/or a specific pH adjuster in the formula and/or the system.

A first treatment of the formula and/or the system may be applied to the affected area of the skin of the user. The formula, the system and/or the affected area may be essentially moisture free. The formula and/or the system may be rubbed onto the skin at the affected area of the user. The urushiol, the chemical irritants and/or the microbiological irritants at the affected area and in the tissue under the affected area may interact with the formula and/or the system and/or may create a lather with the formula and/or the system. The skin at the affected area may be scrubbed with the formula and/or the system. The nonoxynol-9, C12-C15 Pareth-9 and/or sodium cocoyl sarcosinate may clean the urushiol, the chemical irritants and/or the microbiological irritants from the affected area of the skin of the user. As a result, the urushiol, the chemical irritants and/or the microbiological irritants may be separated from the skin at the affected area by one or more abrasive agents of the formula and/or the system.

The urushiol, the chemical irritants and/or the microbiological irritants may bond to one or more surfactants in the cream. Further, the cleansing agent of the formula and/or the system may withdraw and/or may extract the urushiol, the chemical irritants and/or the microbiological irritants from the tissue under the affected area of the skin of the user. As a result, the urushiol, the chemical irritants and/or the microbiological irritants at the affected area and/or in the tissue under the affected area may be bonded to the formula, the system, one or more surfactants and/or the cleansing agent. The formula and/or the system may displace the urushiol, the chemical irritants and/or the microbiological irritants from attachment sites on the affected area of the skin of the user.

The first treatment of the formula and/or the system may be rinsed from the affected area with a solvent, such as, for example, water and/or the like. The urushiol, the chemical irritants and/or the microbiological irritants which is bonded to the formula and/or the system may be rinsed from and/or carried away from the affected area with the solvent. The urushiol, the chemical irritants and/or the microbiological irritants may be removed from the affected area and from the tissue under the affected area of the skin of the user. As a result, the formula and/or the system may treat the affected area of the skin and/or the urushiol induced contact dermatitis of the user. The zinc oxide and one or more surfactants ay protect the skin of the user from additional absorption of the urushiol, the chemical irritants and/or the microbiological irritants.

A second treatment of the formula and/or the system may be applied to the affected area to remove any remaining urushiol, chemical irritants and/or microbiological irritants at the affected area and/or in the tissue under the affected area of the skin of the user. The formula and/or the system may form the lather with the remaining urushiol, the chemical irritants and/or the microbiological irritants. The urushiol, the chemical irritants and/or the microbiological irritants may bond to the formula, the system, the cleansing agent and/or one or more surfactants. The second treatment of the formula and/or the system may be rinsed from the skin of the user with the solvent. The second treatment of the formula and/or the system may remove the remaining urushiol, the remaining chemical irritants and/or the remaining microbiological irritants from the affected area and/or the tissue under the affected area of the skin of the patient. The formula or the system and the remaining urushiol, the remaining chemical irritants or the remaining microbiological irritants may protect the skin of the user from additional absorption of the urushiol, the chemical irritants and/or the microbiological irritants. As a result, the formula and/or the system may treat the affected area of the skin and/or the urushiol induced contact dermatitis of the user.

Additional treatments of the formula and/or the system may be applied to the affected area to remove any and all of the urushiol, the chemical irritants and/or the microbiological irritants from the affected area of the skin and/or in the tissue under the affected area of the skin of the user. Any remaining urushiol, chemical irritants and/or microbiological irritants may bond to the formula, the system, the cleansing agent and/or one or more surfactants. The additional treatments of the formula and/or the system, the urushiol, the chemical irritants and/or the microbiological irritants may be rinsed from the affected area of the skin of the patient. The urushiol, the chemical irritants and/or the microbiological irritants may be removed from the affected area in the skin and/or in the tissue under the affected area of the skin of the patient. As a result, the urushiol induced contact dermatitis may be treated and/or may be terminated by applying one or more treatments of the formula and/or the system to the affected area of the skin of the user. The present invention should not be deemed as limited to the embodiments of a specific number of additional treatments of the formula and/or the system applied to the affected area of the skin of the user for treating the urushiol induced contact dermatitis, the chemical irritants and/or the microbiological irritants.

The formula and/or the system may treat urushiol induced contact dermatitis, chemical irritants and/or microbiological irritants with the formula and/or the system which may be applied to the affected area of skin of the user. The formula and/or the system may have one or more surfactants and/or the cleansing agent, such as, for example, sodium cocoyl sarcosinate to treat the urushiol induced contact dermatitis, the chemical irritants and/or the microbiological irritants on the skin of the user. Further, the formula and/or the system may have one or more abrasive agents, such as, for example, polyethylene granules and/or silica to separate and/or to dislodge the urushiol, the chemical irritants and/or the microbiological irritants from the affected area of the skin of the user. The urushiol, the chemical irritants and/or the microbiological irritants may be removed from the affected area and/or withdrawn from the tissue under the affected area by one or more abrasive agents, the cleansing agent and/or one or more surfactants in the cream. The formula, the system, the urushiol, the chemical irritants and/or the microbiological irritants may be rinsed away from, may be removed from and/or may be withdrawn from the affected area of the skin of the user and/or the tissue under the affected area of the skin of the user. As a result, the urushiol induced contact dermatitis, the chemical irritants and/or the microbiological irritants may be treated and/or may be terminated by the formula, the system, the cleansing agent and/or one or more surfactants.

It should be understood that various changes and modifications to the presently preferred embodiments described herein will be apparent to those skilled in the art. Such changes and modifications may be made without departing from the spirit and scope of the present invention and without diminishing its attendant advantages. It is, therefore, intended that such changes and modifications be covered by the appended claims.

We claim:

1. A formula for treating dermatitis on an area of skin of a user wherein the area of the skin is affected with urushiol wherein the formula is applied to the area of the skin of the user, the formula comprising:
a cleansing agent to bind to the urushiol at the area of the skin of the user;
a first surfactant to remove the urushiol from the area of the skin of the user; and
a first abrasive agent to separate the urushiol from the skin of the user wherein the formula and the urushiol are rinsed from the area of the skin of the user.

2. The formula of claim 1 wherein the first surfactant is nonoxynol-9 or C12-15 pareth-9.

3. The formula of claim 1 wherein the cleansing agent is sodium cocoyl sarcosinate.

4. The formula of claim 1 further comprising:
a second surfactant.

5. The formula of claim 1 further comprising:
a photo-stabilizer.

6. The formula of claim 1 wherein the first abrasive agent is silica or polyethylene granules.

7. The formula of claim 1 further comprising:
a second abrasive agent.

8. The formula of claim 1 further comprising:
a zinc oxide.

9. A system for treating an area of skin of a user wherein the area of the skin is affected with urushiol and further wherein the formula is applied to the area of the skin of the user to remove the urushiol therefrom, the system comprising:
a plurality of surfactants to bond to the urushiol wherein one of the plurality of surfactants is a cleansing agent;
an abrasive agent to separate the urushiol from the skin of the user at the area affected with the urushiol; and
a photo-stabilizer to protect one of the plurality of surfactants from photo-degradation wherein the formula and the urushiol are washed from the area of the skin of the user.

10. The formula of claim 9 wherein one of the plurality of surfactants is nonoxynol-9 or C12-15 pareth-9.

11. The system of claim 9 wherein the cleansing agent is sodium cocoyl sarcosinate.

12. The system of claim 9 wherein the abrasive agent is silica or polyethylene granules.

13. The system of claim 9 wherein the photo-stabilizer is a zinc oxide.

14. A method for treating dermatitis on an area of skin of a user wherein the area of the skin of the user is affected with urushiol, the method comprising the steps of:
providing a formula to remove the urushiol from the area of skin of the user wherein the formula has a first surfactant, a first abrasive agent and a cleansing agent therein;
applying a first treatment of the formula to the area of the skin of the user;
allowing the cleansing agent to bond to the urushiol on the area of the skin of the user; and
removing the formula and the urushiol from the area of the skin of the user.

15. The method of claim 14 further comprising the step of:
adding a photo-stabilizer to the formula.

16. The method of claim 14 further comprising the step of:
adding a second surfactant or a second abrasive agent to the formula.

17. The method of claim 14 further comprising the step of:
applying a second treatment of the formula to the area of the skin of the user.

18. The method of claim 14 further comprising the step of:
separating the urushiol from the area of the skin of the user with the abrasive agent.

19. The method of claim 14 further comprising the step of:
protecting the first surfactant from photo-degradation.

20. The method of claim 14 wherein the cleansing agent is sodium cocoyl sarcosinate.
